(19) United States
(12) Patent Application Publication
(10) Pub. No.: US 2003/0143263 A1
(43) Pub. Date: Jul. 31, 2003

(54) Dry cleansing wipe with improved rinsibility

(75) Inventors: Catherine Durden, Midland Park, NJ (US); Makonnen Payne, Bronx, NJ (US)

Correspondence Address: CHARLES N.J. RUGGIERO, ESQ., OHLANDT, GREELEY, RUGGIERO & PERLE, L.L.P., 10 th FLOOR, ONE LANDMARK SQUARE, STAMFORD, CT, 06901-2682, US

(73) Assignee: PLAYTEX PRODUCTS, INC.

(21) Appl. No.: 10/356,352

(22) Filed: Jan. 30, 2003

Related U.S. Application Data
(63) Continuation-in-part of application No. 10/060,962, filed on Jan. 30, 2002.

(51) Int. Cl. A47K 7/03; A61K 9/70; B32B 27/12; B32B 27/04

(52) U.S. Cl. 424/443; 15/104.93; 442/123

(57) ABSTRACT

A disposable, single use dry cleansing cloth or wipe is provided. The cleansing cloth has a water insoluble substrate and a deposited cleansing composition having a lathering surfactant. The cleansing composition is deposited in an amount such that the cleansing composition rinses out after several re-wettings of said cloth.

### BACKGROUND OF THE INVENTION

1. Field of the Invention

This application is a Continuation-In-Part of U.S. application Ser. No. 10/060,962, filed on Jan. 30, 2002.

The present invention relates generally to a substantially dry cleansing cloth or wipe that is activated by wetting. More particularly, the present invention relates to a cleansing cloth having a cleanser that has improved rinsibility. The present invention further provides that the cleansing cloth has a cleanser that rinses from the cloth after a prescribed number of re-wettings.

2. Description of the Prior Art

Traditional methods of bathing require a multi-step process of depositing cleansing composition from a bar or a bottle onto a substrate, and then using the cleansing composition-laden substrate as a vehicle for applying the cleansing composition to the body for washing. Alternately, a cleansing composition may be dispensed into the hand and then applied to the body. These methods of application, cumbersome even for adults, are ill-suited to bathing a baby because such methods require removal of one hand from the baby in order to dispense the cleansing composition onto the hand or cloth or onto the body.

Dry single use cleansing cloths afford the convenience of providing a wash cloth and cleansing composition in one simple step. Single use cleansing cloths eliminate the need for cumbersome bars, bottles or jars of cleansing composition. Thus, single use cloths are ideally suited to use in a baby's bath. While the cleansing wipe of the present invention is described, by way of example, for use in bathing a baby, the wipe may be used for any cleansing need.

Disposable towelettes with imbedded cleansing composition are known. U.S. Pat. No. 5,980,931 to Fowler et al. describes a substantially dry cleansing cloth having a water insoluable substrate with both a cleansing and a conditioning agent deposited thereon.

U.S. Pat. No. 6,132,746 to Hasenoehrl et al. describes a substantially dry, disposable cleansing cloth having a cleansing agent with improved moisturization properties. A conditioning component added to the cleansing agent acts to deposit an amount of conditioner to the skin greater than about 2.5 micrograms/cm².

U.S. Pat. No. 6,190,678 B1 to Hasenoehrl et al. describes a substantially dry, disposable cleansing cloth with an improved method for deposition of conditioning agents onto the skin. The conditioning agent has a lipid hardness value of greater than about 0.02 kg.

While existing disposable cleansing cloths provide bathing convenience, they do not provide for the deposit of an amount of cleansing composition to a water insoluble substrate that is optimized for rinsing out after a prescribed number of re-wet cycles. Accordingly, the prior art does not provide that the substrate can then be used for rinsing the cleansing composition from the skin.

Therefore, a need exists for a cleansing cloth having a cleansing composition with the added convenience of easily rinsing the cleansing composition out of the cloth after several submersions into a bath so that the cloth can then be used as a rinsing cloth.

### SUMMARY OF THE INVENTION

It is an object of the present invention to provide a substantially dry cleansing cloth having a water insoluble substrate and a cleansing composition containing a lathering surfactant deposited thereon.

It is another object of the present invention to provide such a substantially dry cleansing cloth that is disposable and intended for single use.

It is yet another object of the present invention to provide such a substantially dry cleansing cloth on which a cleansing composition is applied in discrete areas.

It is still another object of the present invention to provide a disposable, substantially dry cleansing cloth in which the cleansing composition is rinsed from the cloth after a prescribed number of rinses.

It is a further object of the present invention to provide a disposable, substantially dry cleansing cloth that may be used to rinse the cleansing composition from the body.

It is a still further object of the present invention to provide a disposable, substantially dry cleansing cloth in which the ratio of cleanser to fabric substrate in a dry state is in the range about 1:1 to 1:8, with 1:3 being the preferred range.

It is another object of the present invention to provide such a cleansing cloth having a cleansing composition that moisturizes and conditions the skin.

It is still another object of the present invention to provide such a cleansing cloth having a cleansing composition with a moisturizer system that contains a milk protein.

It is a further object of the present invention to provide such a cleansing cloth having a cleansing composition that promotes a calming effect and overall enhancement of mood.

It is a still further object of the present invention to provide such a cleansing cloth having a cleansing composition that has an aromatherapy component for promoting a calming effect and overall enhancement of mood.

These and other objects and advantages of the present invention are achieved by a disposable, single use substantially dry cloth onto which a cleanser is deposited in discrete areas by striping, dotting or printing onto the substrate surface. The patterned application of the cleanser enables a controlled amount of deposition of cleanser on each cloth, thereby controlling the amount of lathering action per cloth. The cleanser is deposited in an amount that is optimized to rinse out of the cloth after a prescribed number re-wettings. The cleansing composition may have an aromatherapy system and a moisturizer system. The cloth can then be used to rinse the cleanser from the body.

### DETAILED DESCRIPTION OF THE INVENTION

The present invention provides a disposable, single use dry cloth or wipe that has a cleansing composition deposited thereon. The cleansing composition contains a surfactant that provides lathering properties. The approximate composition of the cleansing composition is about 95 wt. % cleansers and other ingredients and approximately 5 wt. % residual water.

The cleansing composition has one or more surfactants for providing lather. Lathering surfactants that can be used in the present invention may include one or more imidazoline derivatives, fatty sulfosuccinate esters or amides, ethoxylated sorbitan esters, alkylamido alkylamines, betaines, alkoxylated alcohols, phosphorous derivatives, quaternary ammonium compounds, ethoxylated alkyl phenols, alkyl ether sulfates, or any combination thereof. Surfactants are preferably present in an amount about 10 wt. % to about 100 wt. % of the total weight of the cleansing composition. The more preferred range of surfactants is about 50 wt. % to about 100 wt. %, with about 90 wt. % being the most preferred amount, based on the total weight of the cleansing composition. Preferred surfactants that can be used in the present cleansing composition include, but are not limited to, PEG 80 glyceryl cocoate, PEG 30 glyceryl cocoate, cocamidopropyl betaine, decyl glucoside, sodium laureth sulfate, or any combinations thereof.

The cleansing composition may also have a moisturizer or moisturizer system. The moisturizer system, when present, has a hydrolyzed milk protein and a moisturizer. Suitable moisturizers that can be used in the moisturizer system may include, for example, one or more amidoamine salts, lactic acid salts, sunflowerseed amidopropyl dimethylamine lactate (MACKALENE 1216), isostearamidopropyl morpholine lactate (MACKALENE 426), sodium C₁₂-C₁₅ alkoxypropyl iminodiproprionate and laureth-12 sulfosuccinate (CETYLSIL NS), cocoglucoside and glyceryl oleate (LAMESOFT PO 65), casteryl maleate (CERAPHYL RMT), olive oil, glycerin, aloe, or any combinations thereof. Preferably, the moisturizer system includes a sunflowerseed amidopropyl dimethylamine lactate (MACKALENE 1216), glycerin, or any combinations thereof.

As stated above, the moisturizer system of the cleansing composition also has at least one milk protein and/or milk protein hydrolysate. The milk protein serves as a cationic conditioning agent for the skin. The milk protein compound has been found to enhance the moisturizing of the skin. Suitable milk protein compounds for use in the present invention include, for example, hydrolyzed milk protein, hydrolyzed milk protein derivative, or any combinations thereof. Suitable hydrolyzed milk protein derivatives include, for example, palmitoyl derivative and quaternary ammonium salt derivative. Preferably, the milk protein used in the present cleansing composition is a hydrolyzed milk protein sold under the tradename Hydrolactin 2500® by Croda, Inc.

To achieve enhanced moisturizing properties, the moisturizer system, when present, is present in the composition of the present invention in an amount about 0.01 wt. % to about 10 wt. % of the total weight of the composition. Preferably, the moisturizer system is present in an amount about 4 wt. % to about 6 wt. %. The milk protein is preferably present in the composition in an amount about 0.001 wt. % to about 2 wt. % while the moisturizer is preferably present in an amount about 0.3 wt. % to about 1.0% of the total weight of the composition.

The cleansing composition preferably has a fragrance. The fragrance is preferably present in an amount about 0.05 wt. % to about 2.5 wt. % based on the total weight of the cleansing composition. More preferably, the fragrance is present in an amount about 1.2 wt. % based on the total weight of the cleansing composition.

The fragrance of the cleansing composition may further have an aromatherapy component. Aromatherapy has been used for centuries as a way to promote physical and mental well being. Essential oils extracted from flowers, herbs, spices, woods, and fibers, when inhaled, are believed to have subtle effects on a person's mind and emotions. In addition, it is believed that when the essential oils penetrate the skin and enter the bloodstream and immune system, they work in a physiological manner to promote healing.

Essential oils are highly scented droplets found in minute quantities in the flowers, stems, leaves, roots and barks of aromatic plants. They are highly fluid and exceptionally volatile and potent. Because of their high potency, very small amounts of essential oil are needed to experience their benefits.

Essential oils are complex mixtures of different organic molecules, such as terpenes, alcohols, esters, aldehydes, ketones and phenols. It is believed that it is the interaction between each and every component and/or molecule that gives an essential oil its particular character and unique therapeutic properties. Therefore, the use of one or more essential oils in the present composition not only provides a calming effect, it may also provide one or more beneficial healing effects.

A variety of essential oils may be used for the present invention. Suitable essential oils for use in the cleansing composition include, for example, Abies Sibirica oil, amyris balsamifera oil, anise (Illicium Verum) oil, balm mint (Melissa Officinalis) oil, basil (Ocimum Basilicum) oil, bay (Pimenta Acris) oil, bee balm (Monarda Didyma) oil, bergamot (Citrus Aurantium Bergamia) oil, birch (Betula Aba) oil, bitter orange (Citrus Aurantium Amara) oil, cabbage rose (Rosa Centifolia) oil, Calendula Officinalis oil, California nutmeg (Torreya Californica) oil, Camellia Sinensis oil, Capsicum Frutescers oleoresin, caraway (Carum Carvi) oil, cardamon (Elettaria Cardamomum) oil, cedarwood (Cedrus Atlantica) oil, Chamaecyparis Obtusa oil, chamomile (Anthemis Nobilis) oil, cinnamon (Cinnamomum Cassia) oil, citronella (Cymbopogon Nardus) oil, clary (Salvia Sclarea) oil, clove (Eugenia Caryophyllus) oil, cloveleaf (Eugenia Caryophyllus) oil, coriander (Coriandrum Sativum) oil, coriander (Coriandrum Sativum) seed oil, Cyperus Esculentus oil, cypress (Cupressus Sempervirens) oil, Eucalyptus Citriodora oil, Eucalyptus Globulus oil, fennel (Foeniculum Vulgare) oil, gardenia florida oil, Geranium Maculatum oil, ginger (Zingiber Officinale) oil, gold of pleasure (Camelina Sativa) oil, grapefruit (Citrus Grandis) oil, hops (Humulus Lupulus) oil, hypericum perforatum oil, hyptis suaveolens oil, indigo bush (Dalea Spinosa) oil, jasmine (Jasminum Officinale) Oil, Juniperus Communis oil, Juniperus Virginiana oil, Labdanum (Cistus Labdaniferus) oil, laurel (Laurus Nobilis) oil, lavandin (Lavandula Hybrida) oil, lavender (Lavandula Angustifolia) oil, lemon (Citrus Medica Limonum) oil, lemongrass (Cymbopogon Schoenanthus) oil, leptospermum scoparium oil, lime (Citrus Aurantifolia) oil, linden (Tilia Cordata) oil, litsea cubeba oil, lovage (Levisticum Officinale) oil, mandarin orange (Citrus Nobilis) oil, massoy bark oil, matricaria (Chamomilla Recutita) oil, Moroccan chamomile Oil, musk rose (Rosa Moschata) oil, myrrh (Commiphora Myrrha) oil, myrtle (Myrtus Communis) oil, norway spruce (Picea Excelsa) oil, nutmeg (Myristica Fragrans) oil, olax dissitiflora oil, olibanum, opoponax oil, orange (Citrus Aurantium Dulcis) flower oil, orange (Citrus Aurantium Dulcis) oil, palmarosa (Cymbopogon Martini) oil, parsley (Carum Petroselinum) seed oil, passionflower (Passiflora Incarnata) oil, patchouli (Pogcstemon Cabin) oil, pelargonium graveolens oil, pennyroyal (Mentha Pulegium) oil, peppermint (Mentha Piperita) oil, pine (Pinus Palustris) oil, pine (Pinus Palustris) tar oil, pine (Pinus Pinea) kernel oil, pine (Pinus Pumiho) oil, pine (Pinus Sylvestris) cone oil, rosemary (Rosmarinus Officinalis) oil, rose oil, rosewood (Aniba Rosseodora) oil, rue (Ruts Graveolens) oil, sage (Salvia Officinalis) oil, sambucus nigra oil, sandalwood (Santalum Album) oil, sandarac (Callitris Quadrivalvis) gum, sassafras officinale oil, sisymbrium ino oil, spearmint (Mentha Viridis) oil, sweet marjoram (Origanum Majorana) oil, sweet violet (Viola Odorata) oil, tar oil, thuja occidentalis oil, thyme (Thymus Vulgaris) oil, vetiveria zizanoides oil, wild mint (Mentha Arvensis) oil, ximenia americana oil, yarrow (Achillea Millefolium) Oil, ylang yang (Cananga Odorata) oil, or any combinations thereof.

The preferred essential oils for use in the present invention are lavendin and chamomile. Lavendin, a botanical variety of lavender, is used for promoting relaxation and calming effect. Chamomile is used for promoting calm and relaxation.

When present, the one or more essential oils are present in an amount about 0.00001 wt. % to about 1 wt. %, based on the total weight of the composition. Preferably, the one or more essential oils when present, are present in an amount about 0.00005 wt. % to about 0.1 wt. %, and more preferably about 0.0001 wt. % to about 0.01 wt. % of the total weight of the composition.

In particular, the aromatherapy component of the cleansing composition includes lavendin and chamomile oils. The aromatherapy component lavendin is present in an amount about 0.00005 wt. % to about 0.05 wt. %, and preferably in an amount about 0.009 wt. % of the total weight of the composition. The aromatherapy component chamomile is present in an amount about 0.00005 wt. % to about 0.05 wt. %, and preferably in an amount about 0.00015 wt. % of the total weight of the composition.

Water is preferably present in an amount greater than about 0 wt. % to about 10 wt. %. More preferably, water is present in an amount about 5 wt. % based on the total weight of the cleansing composition.

The present compositions may also have one or more preservatives. The preservatives act to mitigate against unwanted mold or bacteria in the finished product. Preservatives that can be used in the present cleansing compositions include one or more parabens including methylparaben and propyparaben; organic acids and their derivatives including citric acid, sorbic acids, malic acids; idophors; quaternary ammonium compounds; urea derivatives; isothiazolinones; alkyl substituted amino acids; or any combinations thereof. Preferred preservatives include imidazolidinyl urea, methylparaben, propylparaben, or any combinations thereof. Preservatives are preferably present in an amount about 0.05 wt. % to about 5.0 wt. % of the total weight of the cleansing composition. The more preferred range of preservatives is about 0.1 wt. % to about 3.0 wt. %, with 2.0 wt. % being the most preferred amount, based on the total weight of the cleansing composition.

In addition, the cleansing composition preferably has a pH adjuster. One such pH adjuster that can be used in the present cleansing composition is citric acid. The pH adjuster is preferably present in an amount about 0.01 wt. % to about 1.0 wt. % based on the total weight of the cleansing composition. More preferably, the pH adjuster is present in an amount about 0.4 wt. % based on the total weight of the cleansing composition.

The cleansing composition may also include one or more coloring agents. Non-limiting examples of coloring agents include: dye, food coloring or cosmetic pigments. The coloring agent or agents, when present, are present in an amount about 0.0001 wt % to about 0.05 wt %.

The cleansing composition may have a skin conditioner. The skin conditioner acts to nourish and soothe the skin. Skin conditioners that can be used in the present cleansing compositions include, but are not limited to, one or more lanolins, polyols including glycerin and sorbitol, botanical derivatives including aloe, vitamins including vitamin E, silicones including dimethicone, quaternary surfactants, esters, mineral oils, petrolatums, C7-C40 branched chain hydrocarbons, C1-C30 alcohol esters of C1-C30 carboxylic acids, C1-C30 alcohol esters of C2-C30 dicarboxylic acids, monoglycerides of C1-C30 carboxylic acids, diglycerides of C1-C30 carboxylic acids, triglycerides of C1-C30 carboxylic acids, ethylene glycol monoesters of C1-C30 carboxylic acids, ethylene glycol diesters of C1-C30 carboxylic acids, propylene glycol monoesters of C1-C30 carboxylic acids, propylene glycol diesters of C1-C30 carboxylic acids, C1-C30 carboxylic acid monoesters and/or polyesters of sugars, polydialkylsiloxanes, polydiarylsiloxanes, polyalkarylsiloxanes, cylcomethicones having 3 to 9 silicon atoms, vegetable oils, hydrogenated vegetable oils, polypropylene glycol C4-C20 alkyl ethers, di C8-C30 alkyl ethers, or any combinations thereof.

The following is an example of the cleansing composition of the preferred embodiment of the present invention.

### EXAMPLE 1

| Ingredient | w/w % |
|---|---|
| Water | 5.00 |
| Surfactant | |
| PEG 80 Glyceryl Cocoate (and) | 51.65 |
| PEG 30 Glyceryl Cocoate | |
| Cocamidopropyl Betaine (30%) | 12.63 |
| Decyl Glucoside (50%) | 13.61 |
| Sodium Laureth Sulfate (26%) | 13.61 |
| Fragrance | 1.25 |
| Preservative | |
| Imidazolidinyl Urea and | 1.06 |
| Propylene Glycol (solvent) | |
| Methylparaben | 0.49 |
| Propylparaben | 0.34 |
| PH Adjuster | |
| Citric Acid | 0.36 |

The cleansing composition is deposited on the substrate cloth or wipe in an amount such that the cleansing composition rinses out of the cloth after several re-wettings. The substrate is re-wet when it is substantially moistened, covered, or saturated with water or other liquid. In the preferred embodiment of the present invention, the cleansing composition rinses out of the cloth after 3 to 5 re-wettings. Once the cleansing composition is rinsed from the cloth, the cloth may then be used to rinse the cleansing composition from the body or substrate upon which the cloth or wipe has been used.

The cleansing composition is applied to the substrate in an amount that is optimized to rinse from the substrate after 3 to 5 re-wettings. Preferably about 0.5 g to about 4.0 g of cleansing composition is applied to approximately 3.1 g to about 3.8 g of fabric. More preferably, approximately 1.25 g of cleansing composition is applied to one 6.5″×7.0″ washcloth, which weighs approximately 3.5 g. The approximate ratio of cleansing composition to fabric in a wipe or cloth of the present invention is in the range about 1:1 to about 1:8. Preferably, the approximate ratio of cleansing composition to fabric in a wipe or cloth of the present invention is in the range about 1:3.

The cleaning composition is deposited on the substrate by one or a combination of methods to produce discrete, patterned areas. The patterned application of the cleansing composition enables a controlled amount of deposition of cleansing composition on each cloth, thereby controlling the amount of lathering action per cloth. The application in discrete areas may be accomplished by striping, dotting or printing the cleansing composition onto the substrate surface.

Striping, dotting or printing the cleansing composition onto the substrate may be accomplished by spraying the cleansing composition onto the substrate to coat the substrate. During the spraying process, a roll of substrate is unwound beneath a series of spraying heads. The spraying heads are adjusted to deliver the specified amount of cleansing composition onto the substrate. The sprayed substrate is then passed through a drying oven to drive off residual water, and is then either re-wound or cut, folded and placed into suitable packaging.

Alternately, the cleansing composition may be striped, dotted or printed onto the substrate by slot coating. In the slot coating process, the cleansing composition is forced through a slot onto the surface of the substrate, thereby producing a substrate patterned with cleansing composition. During the slot coating process, the roll of substrate is unwound and passed before or beneath a metal slot through which a specified amount of cleaning composition is delivered. Following coating, the substrate then passes through a drying oven to drive off residual water, and is then either re-wound into a roll or cut, folded and placed into suitable packaging.

The cleansing composition may also be applied by a knife over blade process. In this process, the cleansing composition is flooded onto the substrate surface. The excess cleansing composition is then removed using a blade. After coating, the substrate passes through a drying oven to drive off residual water, and is then either re-wound or cut, folded and placed into suitable packaging.

The cleansing cloth substrate is formed of a nonwoven fabric that is uniformly free of gaps. Nonwoven fabric is made of fibers that are formed into a sheet rather than woven into a fabric. The nonwoven fabric substrate can be formed of a single layer or multiple layers of fabric. In the preferred embodiment, the nonwoven substrate is a single layer of needlepunched fabric with a basis weight of about 100 grams per square meter that has about 25% to about 35% lyocell fiber by weight, and about 65% to about 75% polyester fiber by weight. The cross directional tensile strength of the dry nonwoven fabric is a minimum of 2000 g/in. The needlepunched fabric may also be constructed of varying combinations (totaling 100%) of lyocell, polyester, rayon, or polypropylene to optimize the strength, thickness and feel of the fabric. In an alternate embodiment, the substrate may be a single layer of spun laced fabric containing about 50% rayon and about 50% polyester by weight. Methods of making nonwoven substrates are well known in the art and include air-laying, water-laying, meltblowing, spun lacing or carding a layer of natural and synthetic fabric that is then subjected to bonding.

The present invention has been described with particular reference to the preferred embodiments. It should be understood that the foregoing descriptions and examples are only illustrative of the present invention. Various alternatives and modifications thereof can be devised by those skilled in the art without departing from the spirit and scope of the present invention. Accordingly, the present invention is intended to embrace all such alternatives, modifications, and variations that fall within the scope of the appended claims.

What is claimed is:

1. A disposable, single use dry cloth having a cleanser deposited thereon comprising:
a water insoluble substrate; and
a cleansing composition having a lathering surfactant,
wherein said cleansing composition is deposited in an amount such that it rinses out after several re-wettings of said cloth.

2. The cloth of claim 1, wherein said cleansing composition is deposited in a pattern.

3. The cloth of claim 2, wherein said pattern is striped.

4. The cloth of claim 2, wherein said pattern is dotted.

5. The cloth of claim 1, wherein the ratio of said cleansing composition to said substrate is in the range about 1:1 to 1:8.

6. The cloth of claim 1, wherein the ratio of said cleansing composition to said substrate is in the range about 1:3.

7. The cloth of claim 1, wherein the cloth can be used to rinse said cleansing composition from a body.

8. The cloth of claim 1, wherein said cleansing composition is applied to said substrate by spraying.

9. The cloth of claim 1, wherein said cleansing composition is applied to said substrate by slot coating.

10. The cloth of claim 1, wherein said cleansing composition is applied to said substrate by a knife over blade process.

11. The cloth of claim 1, wherein said cleansing composition is deposited on said substrate in an amount about 0.5 g to about 4.0 g.

12. The cloth of claim 1, wherein said cleansing composition is deposited on said substrate in an amount about 0.5 g to about 2.0 g in a dry state.

13. The cloth of claim 1, wherein said cleansing composition is deposited to said substrate in an amount about 0.5 g to about 2.0 g of said cleansing composition to about 3.1 g to about 3.8 g of said substrate.

14. The cloth of claim 1, wherein said cleansing composition is deposited to said substrate in an amount about 1.25 g of said cleansing composition to about 3.5 g of said substrate.

15. The cloth of claim 1, wherein said substrate is a needlepunched fabric.

16. The cloth of claim 15, wherein said needlepunched fabric has about 35% lyocell and about 65% polyester.

17. The cloth of claim 1, wherein said cleansing composition further comprises water and at least one lathering surfactant.

18. The cloth of claim 1, wherein said cleansing composition further comprises a moisturizer system.

19. The cloth of claim 18, wherein said moisturizer system comprises one or more moisturizers selected from the group consisting of amidoamine salt, lactic acid salt, sunflowerseed amidopropyl dimethylamine lactate, isostearamidopropyl morpholine lactate, sodium C₁₂-C₁₅ alkoxypropyl iminodiproprionate, laureth-12 sulfosuccinate, cocoglucoside, glyceryl oleate, casteryl maleate, olive oil, glycerin, and any combinations thereof.

20. The cloth of claim 18, wherein moisturizer system comprises a moisturizer selected from the group consisting of sunflowerseed amidopropyl dimethylamine lactate, glycerin, and any combinations thereof.

21. The cloth of claim 18, wherein said moisturizer system includes a milk protein.

22. The cloth of claim 21, wherein said milk protein is quaternium-79 hydrolyzed milk protein.

23. The cloth of claim 1, wherein said cleansing composition further comprises an aromatherapy system.

24. The cloth of claim 23, wherein said aromatherapy system includes at least one essential oil.

25. The cloth of claim 24, wherein said at least one essential oil is lavendin.

26. The cloth of claim 24, wherein said at least one essential oil is chamomile.

27. The cloth of claim 1, wherein said cleansing composition further comprises water and at least one preservative.

28. The cloth of claim 17, wherein said cleansing composition further comprises a fragrance.

29. The cloth of claim 1, wherein said cleansing composition further comprises a pH adjuster.

30. A disposable, single use dry cloth having a cleanser deposited thereon comprising:
a water insoluble substrate; and
a cleansing composition having a lathering surfactant and an aromatherapy system,
wherein said cleansing composition is deposited in an amount such that it rinses out after several re-wettings of said cloth.

31. A disposable, single use dry cloth with a cleanser deposited thereon comprising:
a water insoluble substrate; and
a cleansing composition having a lathering surfactant, said cleansing composition being deposited in a pattern on said substrate;
wherein said cleansing composition is deposited in an amount such that it rinses out after several re-wettings of said cloth.

32. The cloth of claim 31, wherein said cleansing composition further comprises water and at least one preservative.

33. The cloth of claim 31, wherein said cleansing composition further comprises an aromatherapy system.

34. A disposable, single use dry cloth with a cleanser deposited thereon comprising:
a water insoluble substrate; and
a cleansing composition having a lathering surfactant and an aromatherapy system, said cleansing composition being in a pattern on said substrate;
wherein said cleansing composition is in an amount such that it rinses out after several re-wettings of said cloth.

35. A method of manufacturing a single use dry cloth with a cleanser deposited thereon comprising:
providing a water insoluble substrate; and
depositing a cleansing composition having a lathering surfactant to said substrate,
wherein said cleansing composition is deposited in an amount such that it rinses out after several re-wettings of said cloth, and
wherein the ratio of said cleansing composition to said substrate is in the range about 1:1 to about 1:8.

36. A method of manufacturing a single use dry cloth with a cleanser deposited thereon comprising:
providing a water insoluble substrate; and
depositing a cleansing composition having a lathering surfactant to said substrate,
wherein said cleansing composition is deposited in an amount such that it rinses out after several re-wettings of said cloth, and
wherein the ratio of said cleansing composition to said substrate is in the range about 1:3.

37. The method of claim 36, wherein said cleansing composition is deposited in a pattern.

38. The method of claim 36, wherein said cleansing composition is applied by spraying.
